(19) United States
(12) Patent Application Publication
(10) Pub. No.: US 2003/0114460 A1
(43) Pub. Date: Jun. 19, 2003

(54) Pharmaceutical conjugates with enhanced pharmacokinetic characteristics

(75) Inventors: Patrick M. Hughes, Aliso Viejo, CA (US); Orest Olejnik, Coto De Caza, CA (US)

Correspondence Address: STOUT, UXA, BUYAN & MULLINS LLP, 4 VENTURE, SUITE 300, IRVINE, CA, 92618, US

(73) Assignee: Allergan Sales, Inc., Irvine, CA

(21) Appl. No.: 10/016,850

(22) Filed: Dec. 14, 2001

(51) Int. Cl. A61K 31/498; A61K 31/16; C07D 241/36

(52) U.S. Cl. 514/249; 514/613; 544/353; 564/161

(57) ABSTRACT

Pharmaceutical conjugates comprising a therapeutic component and an efficacy enhancing component that enhances the pharmacokinetic disposition of the therapeutic component is disclosed. In one embodiment, therapeutic component is joined to an efficacy enhancing component by a linker. In one embodiment, the therapeutic component and the efficacy enhancing component disassociate under physiolocal conditions, preferably near the site where the therapeutic component may exert a therapeutic effect.

### BACKGROUND OF THE INVENTION

The present invention relates to pharmaceutical conjugates with enhanced pharmacokinetics. In particular, the invention relates to pharmaceutical conjugates comprising a therapeutic component (TC) and an efficacy enhancing component (EEC). Preferably, the TC and the EEC are chemically joined together.

A TC includes any chemical entity, such as a compound, an ion, a complex and the like, which is effective to act on and/or bind to receptors and provide a therapeutic effect. The TC may be an agonist, an antagonist, precursors thereof, metabolites thereof and combinations thereof.

A continuing challenge in pharmaceutical sciences is to provide for pharmaceutical conjugates with enhanced pharmacokinetics. The term “enhanced pharmacokinetic” as used herein means an enhancement in permeability, bioavailability, binding and/or sequestration characteristics of the TCs, by itself or bound to another molecule.

The use of drugs to treat the eye presents a particular challenge. For example, topical delivery of drugs to treat the posterior chamber tissues such as the retina, vitreous and posterior uveal tract is virtually impossible. This is primarily due to low corneal and lens permeability and rapid precorneal clearance of instilled drugs results in only a few percent of the applied dose being absorbed into the aqueous humor. Furthermore, the elimination of the aqueous humor from the through normal aqueous humor turnover continuously reduces the aqueous humor concentration of absorbed drug. Additionally the iridolenticular diaphragm prevents drug from reaching the posterior of the eye. Such constraints prevent topically administered drug from reaching the vitreous or retina.

Various techniques have been employed to overcome the difficulties of delivering drugs to the eye. For example, delivery of drugs to the retina, vitreous and uveal tract is typically achieved by high systemic dosing, intra-ocular injections, intra-ocular implants or other heroic measures.

However, these techniques are not very effective. For example, penetration of systemically administered drugs into the retina is severely restricted by the blood-retinal barriers (BRB) for most compounds. Moreover, systemically administered drugs can diffuse into the iris-ciliary body achieving low aqueous humor concentrations. Furthermore, the anterior bulk flow of aqueous humor competes with the posterior diffusion of drugs into the vitreous. As such, extremely low concentrations of drug is available in only the anterior most vitreous and retina. Also, invasive techniques such as injection or implantation may result in retinal detachment, physical damage to the lens, as well as exogenous endophthalmitis.

There continues to be a need for improved drugs which could be used to treat the eye. For example, there is a need to have a drug which has enhanced pharmacokinetics to allow it to be more available at the treatment site.

### SUMMARY OF THE INVENTION

New drugs, i.e. pharmaceutical conjugates, with improved pharmacokinetics are discovered. Preferably, the new pharmaceutical conjugates have improved pharmacokinetics, for example improved bioavailability, in the eye.

In accordance with the invention, the present pharmaceutical conjugates comprise a therapeutic component (TC) and an efficacy enhancing component (EEC). In one embodiment, the EEC has the general formula A:

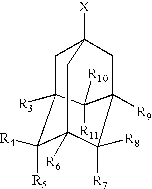

wherein X is an H, a C1-C10 hydrocarbon, or a

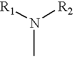

R1, R2, R3, R4, R5, R6, R7, R8, R9, R10 and R11 are independently an H, a C1-C10 hydrocarbon, or a linker.

Further in accordance with the invention, the TC and the EEC are directly joined by a covalent bond. In another embodiment, the TC and the EEC are joined by a linker. Non-limiting examples of linkers include:

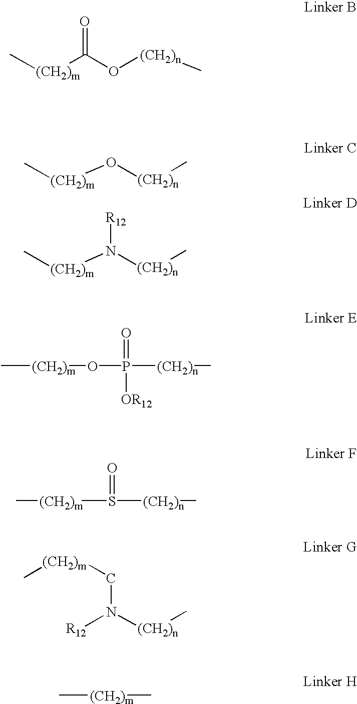

wherein R12 is an H or a C1-C10 hydrocarbon, m=0 to 10, and n=0 to 10. In one embodiment, the EEC is a memantine. In one embodiment, the TC and the EEC disassociate under physiological conditions.

Still further in accordance with the invention, the TC is selected from the group consisting of NMDA antagonists, antibacterials, antihistamines, decongestants, antiinflammatories, antiparasitics, miotics, anticholinergics, adrenergics, antivirals, local anesthetics, antifungals, amoebicidals, trichomonocidals, analgesics, mydriatics, antiglaucoma drugs, carbonic anhydrase inhibitors, ophthalmic diagnostic agents, ophthalmic agents used as adjuvants in surgery, chelating agents, antineoplastics, antihypertensives, muscle relaxants, diagnostics, tyrosine kinase inhibitors and neuroprotectants.

Still further in accordance with the invention, a pharmaceutical conjugate of the present invention may be administered to a subject, for example a human patient, to treat a condition, preferably an eye condition. The routes of administration include topical, oral, rectal, sublingual, nasal, and/or intravenous. Preferably, the pharmaceutical conjugates of the present invention have high bioavailabilities at, near or in the uveal tract, vitreous, retina, choroid and retinal pigmented epithelium after it is administered, for example topically.

Any feature or combination of features described herein are included within the scope of the present invention provided that the features included in any such combination are not mutually inconsistent as will be apparent from the context, this specification, and the knowledge of one of ordinary skill in the art.

Additional advantages and aspects of the present invention are apparent in the following detailed description and claims.

### DETAILED DESCRIPTION OF THE INVENTION

Pharmaceutical conjugates with enhanced pharmacokinetics is provided. Preferably, the pharmaceutical conjugates are ophthalmic pharmaceutical conjugates. In a broad embodiment, the pharmaceutical conjugate comprises a therapeutic component (TC) joined to an efficacy enhancing component (EEC).

Examples of TCs of the present invention include, but are not limited to, NMDA antagonists; antibacterial substances such as beta-lactam antibiotics, such as cefoxitin, n-formamidoylthienamycin and other thienamycin derivatives, tetracyclines, chloramphenicol, neomycin, carbenicillin, colistin, penicillin G, polymyxin B, vancomycin, cefazolin, cephaloridine, chibrorifamycin, gramicidin, bacitracin and sulfonamides; aminoglycoside antibiotics such as gentamycin, kanamycin, amikacin, sisomicin and tobramycin; nalidixic acid and its analogs such as norfloxacin and the antimicrobial combination fluoroalanine/pentizidone, nitrofurazones and analogs thereof; antihistaminics and decongestants such as pyrilamine, chlorpheniramine, tetrahydrazoline, antazoline and analogs thereof; mast-cell inhibitors of histamine release, such as cromolyn; anti-inflammatories such as cortisone, hydrocortisone, hydrocortisone esters, betamethasone, dexamethasone, dexamethasone sodium phosphate, prednisone, methylprednisolone, medrysone, fluorometholone, prednisolone, prednisolone sodium phosphate, triamcinolone, indainethacin, sulindac, its salts and its corresponding sulfides, and analogs thereof; miotics and anticholinergics such as echothiophate, pilocarpine, physostigmine salicylate, diisopropylfluorophosphate, epinephrine, dipivaloylepinephrine, neostigmine echothiopate iodide, demecarim bromide, carbamoyl choline chloride, methacholine, bethanechol, and analogs thereof; mydriatics such as atrophine, homatropine, scopolamine, hydroxyamphetamine, ephedrine, cocaine, tropicamide, phenylephrine, cyclopentolate, oxyphenonium, eucatropine; and the like and mixtures thereof. Other TCs are: antiglaucama drugs, for example, timolol, and especially its maleic salt and R-timolol and a combination of timolol or R-timolol with pilocarpine; other adrenergic agonists and/or antagonists such as epinephrine and an epinephrine complex, or prodrugs such as bitartrate, borate, hydrochloride and dipivefrine derivatives; carbonic anhydrase inhibitors such as acetazolamide, dichlorphenamide, 2-(p-hydroxyphenyl)-thiothiophene-sulfonamide, 6-hydroxy-2-benzothiazolesulfonamide, and 6-pivaloyloxy-2-benzothiazolesulfonamide; antiparasitic compounds and/or anti-protozoal compounds such as ivermectin, pyrimethamine, trisulfapidimidine, clindamycin and corticosteroid preparations; compounds having antiviral activity such as acyclovir, 5-iodo-2′-deoxyuridine (IDU), adenosine arabinoside (Ara-A), trifluorothymidine, interferon, and interferon-inducing agents such as poly I:C; antifungal agents such as amphotericin B, nystatin, flucytosine, natamycin and miconazole; anesthetic agents such as etidocaine cocaine, benoxinate, dibucaine hydrochloride, dyclonine hydrochloride, naepaine, phenacaine hydrochloride, piperocaine, proparacaine hydrochloride, tetracaine hydrochloride, hexylcaine, bupivacaine, lidocaine, mepivacaine and prilocaine; ophthalmic diagnostic agents, such as: (a) those used to examine the retina such as sodium fluorescein, (b) those used to examine the conjunctiva, cornea and lacrimal apparatus, such as fluorescein and rose bengal and (c) those used to examine abnormal pupillary responses such as methacholine, cocaine, adrenaline, atropine, hydroxyamphetamine and pilocarpine; ophthalmic agents used as adjuncts in surgery, such as alpha-chymotrypsin and hyaluronidase; chelating agents such as ethylenediaminetetraacetic acid (EDTA) and deferoxamine; immunosuppressants and anti-metabolites such as methotrexate, cyclophosphamide, 6-mercaptopurine and azathioprine and combinations of the compounds mentioned above, such as antibiotics/antiinflammatories combinations such as the combination of neomycin sulfate and dexamethasone sodium phosphate and combinations concomitantly used for treating glaucoma, for example, a combination of timolol maleate and aceclidine; and the like and mixtures thereof.

In a preferred embodiment, the useful TCs include adrenergic agonists. More preferably, the useful TCs include alpha-adrenergic agonists. Examples of alpha-adrengergic agonists include, but not limited to, adrafinil, adrenolone, amidephrine, apraclonidine, budralazine, clonidine, cyclopentamine, detomidine, dimetofrine, dipivefrin, ephedrine, epinephrine, fenoxazoline, guanabenz, guanfacine, hydroxyamphetamine, ibopamine, indanazoline, isometheptene, mephentermine, metaraminol, methoxamine, methylhexaneamine, metizolene, midodrine, naphazoline, norepinephrine, norfenefrine, octodrine, octopamine, oxymetazoline, phenylephrine, phenylpropanolamine, phenylpropylmethylamine, pholedrine, propylhexedrine, pseudoephedrine, rilmenidine, synephrine, tetrahydrozoline, tiamenidine, tramazoline, tuaminoheptane, tymazoline, tyramine, xylometazoline, and the like and mixtures thereof.

In a still more preferred embodiment, the useful TCs include alpha-2-adrenergic agonists. As used herein, the term “alpha-2 adrenergic agonist” includes chemical entities, such as compounds, ions, complexes and the like, that produces a net sympatholytic response, resulting in increased accommodation, for example, by binding to presynaptic alpha-2 receptors on sympathetic postganglionic nerve endings or, for example, to postsynaptic alpha-2 receptors on smooth muscle cells. A sympatholytic response is characterized by the inhibition, diminishment, or prevention of the effects of impulses conveyed by the sympathetic nervous system. The alpha-2 adrenergic agonists of the invention bind to the alpha-2 adrenergic receptors presynaptically, causing negative feedback to decrease the release of neuronal norepinephrine. Additionally, they also work on alpha-2 adrenergic receptors postsynaptically, inhibiting beta-adrenergic receptor-stimulated formation of cyclic AMP, which contributes to the relaxation of the ciliary muscle, in addition to the effects of postsynaptic alpha-2 adrenergic receptors on other intracellular pathways. Activity at either pre- or postsynaptic alpha-2 adrenergic receptors will result in a decreased adrenergic influence. Decreased adrenergic influence results in increased contraction resulting from cholinergic innervations. Alpha-2 adrenergic agonists also include compounds that have neuroprotective activity. For example, 5-bromo-6-(2-imidozolin-2-ylamino) quinoxaline (Bromonidine) is an alpha-2-adrenergic agonist which has a neuroprotective activity through an unknown mechanism. Without limiting the invention to the specific groups and compounds listed, the following is a list of representative alpha-2 adrenergic agonists useful in this invention: imino-imidazolines, including clonidine, apraclonidine; imidazolines, including naphazoline, xymetazoline, tetrahydrozoline, and tramazoline; imidazoles, including detomidine, medetomidine, and dexmedetomidine; azepines, including B-HT 920 (6-allyl-2-amino-5,6,7,8 tetrahydro-4H-thiazolo[4,5-d]-azepine and B-HT 933; thiazines, including xylazine; oxazolines, including rilmenidine; guanidines, including guanabenz and guanfacine; catecholamines and the like.

Particularly useful alpha-2-adrenergic agonists include quinoxaline components. In one embodiment, the quinoxaline components include quinoxaline, derivatives thereof and mixtures thereof. Preferably, the derivatives of quinoxaline include (2-imidozolin-2-ylamino) quinoxaline. More preferably, the derivatives of quinoxaline include 5-halide-6-(2-imidozolin-2-ylamino) quinoxaline. The “halide” of the 5-halide-6-(2-imidozolin-2-ylamino) quinoxaline may be a fluorine, a chlorine, an iodine, or preferably, a bromine, to form 5-bromo-6-(2-imidozolin-2-ylamino) quinoxaline.

Other useful quinoxaline derivatives are well known. For example, useful derivatives of a quinoxaline include the ones disclosed by Burke et al U.S. Pat. No. 5,703,077. See also Danielwicz et al U.S. Pat. No. 3,890,319. Each of the disclosures of Burke et al and Danielwicz et al is incorporated in its entirety by reference herein.

The quinoxaline and derivatives thereof, for example Brimonidine, are amine-containing and preferably have pKa's of greater than 7, preferably about 7.5 to 9.

Analogs of the foregoing compounds that function as alpha-2 adrenergic agonists also are specifically intended to be embraced by the invention.

Preferably, the alpha-2-adrenergic agonists, for example the ones listed above, are effective toward activating one or more of alpha-2A-adrenergic receptors, alpha-2B-adrenergic receptors and alpha-2D-adrenergic receptors.

Other useful TCs include ocular hypotensive agents (Woodward et al U.S. Pat. No. 5,688,819), pyranoquinolinone derivatives (Cairns et al U.S. Pat. No. 4,474,787), compounds having retinoid-like activities (Chandraratna U.S. Pat. No. 5,089,509), ketorolac/pyrrole-1-carboxylic acids (Muchowski et al U.S. Pat. No. 4,089,969), ofloxacins/benzoxazine derivatives (Hayakawa et al U.S. Pat. No. 4,382,892), memantines (Lipton et al U.S. Pat. No. 5,922,773). Each of the disclosures referred to in the above patents is incorporated in its entirety herein by reference.

A TC, for example the ones listed above, by itself may not have the required pharmacokinetics to be effective when administered topically, systemically, orally, rectally, sublingually or nasally. For example, when administered topically to the cornea, some TCs may not have the proper lipophilicity to penetrate the various layers of the eye to reach the retina. Other TCs may have the proper lipophilicity to penetrate the layers, but are insoluble in the vitreous chamber.

Without wishing to limit the invention to any theory or mechanism of operation, it is believed that when a TC is joined to an EEC of the present invention, it has an increased partition coefficient and/or an increased aqueous solubility. Furthermore, it is believed that the EECs of the present invention bind to the retinal epithelium. The binding of the EECs to the retinal epithelium may cause the TCs to become more bioavailable, in particular at or near the retinal epithelium. Thus, the EECs of the present invention are effective in enhancing the permeability and/or bioavailability of the TC.

For example, memantine may be employed as an EEC in accordance with this invention. The HCl salt of memantine has solubility of 3.5% in a pH 6.5 aqueous solution. The memantine HCl apparent octanol/water partition coefficient is Log 3.0. The high apparent partition and high solubility appear contradictory. However, the charged amine of memantine HCl (pKa 10.27) results in the high solubility while the free base results in the high partitioning of memantine into octanol. As the HCl salt and free base are in equilibrium, permeation of the free base through biologic tissues results. The impact of a high solubility and a high partition coefficient of the free base are a high bioavailability.

Other examples of ECCs of the present invention include, for example, adamantanes, isosteres of adamantanes, derivatives of adamantanes and soft drugs with similar physicochemical and structural properties to adamantanes, preferably adamantaneamines.

In one embodiment, the EEC of the present invention has the general formula A:

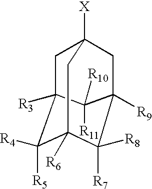

wherein X is an H, a C1-C10 hydrocarbon or

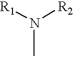

R1, R2, R3, R4, R5, R6, R7, R8, R9, R10 and R11 are independently an H, a C1-C10 hydrocarbon, or a linker.

In a preferred embodiment, R1 and R2 are H's, and R3 is a linker. In a more preferred embodiment, the EEC is a memantine.

In one embodiment, a single TC is joined with more than one EEC, for example two or three EECs. In another embodiment, a single EEC is joined with more than one TC, for example two or three TCs.

In one embodiment, the TC is joined with the ECC directly. In another embodiment, the TC is joined with the ECC through a linker.

Non-limiting examples of linkers include

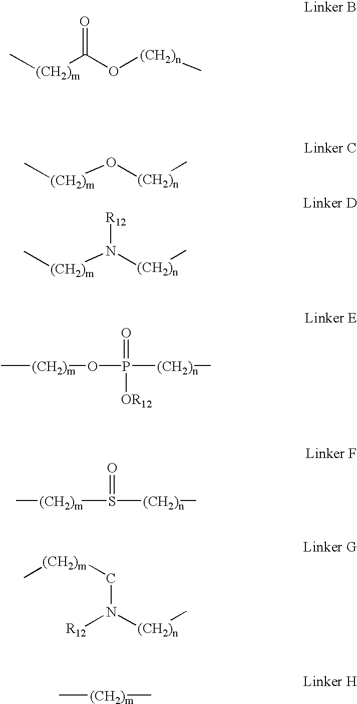

wherein R12 is an H or a C1-C10 hydrocarbon, m=0 to 10, and n=0 to 10. In one embodiment, m is 0 to about 5. In another embodiment, n is 0 to about 5.

In one embodiment, the TC and the EEC making up the pharmaceutical conjugate disassociate under physiological conditions. For example, when a linker such as B above is used, an endogenous esterase may act to cleave the linker, thus disassociating the TC from the EEC. Preferably, the TC and the EEC disassociate at or near the location where the TC is able to exert a therapeutic effect. For example, it is preferable that the TC and the EEC remained joined when it is applied to the surface of the cornea, and disassociate after it enters the vitreous chamber. A conjugate comprising a TC which disassociates to release the TC may be considered a “prodrug” of the TC.

The pharmaceutical conjugates of the present invention may be synthesized according to known techniques. See, for example, Powell et al in Pharmaceutical Research, vol. 8, no. 11, 1991 : 1418-1423; and Tsuzuki et al in Journal of Pharmaceutical Sciences, vol. 83, No. 4, 1994 : 481-484. The pharmaceutical conjugate may be a neutral compound, or a salt. In one embodiment, the counter ions used for forming the salt may be selected from any pharmaceutically acceptable ions, for example sodium, magnesium, chloride, bromide, and iodide.

In one embodiment, the pharmaceutical conjugate comprises a TC covalently joined to an EEC, preferably a memantine. In another embodiment, the pharmaceutical conjugate comprises a TC and an EEC which is covalently joined by a linker. For example, a bromonidine may be joined with a memantine through a linker H (where m=10) to form a “bromonidine conjugate”; or timolol may be joined with memantine through a linker B (where m=0 and n=0) to form a “timolol conjugate”.

The present invention also provides for a pharmaceutical formulation comprising a pharmaceutical conjugate of the present invention. The formulation may include preservative components or components which assist in the preservation of the formulation. The preservative components are selected so as to be effective and efficacious as preservatives in the present formulation, and preferably have reduced toxicity and more preferably substantially no toxicity when the formulation are administered to a human or animal.

Very useful examples of the present preservative components include, but are not limited to oxidative preservative components, for example oxy-chloro components, peroxides, persalts, peracids, and the like, and mixtures thereof. Specific examples of oxy-chloro components useful as preservatives in accordance with the present invention include hypochlorite components, for example hypochlorites; chlorate components, for example chlorates; perchlorate components, for example perchlorates; and chlorite components. Examples of chlorite components include stabilized chlorine dioxide (SCD), metal chlorites, such as alkali metal and alkaline earth metal chlorites, and the like and mixtures therefore. Technical grade (or USP grade) sodium chlorite is a very useful preservative component. The exact chemical composition of many chlorite components, for example, SCD, is not completely understood. The manufacture or production of certain chlorite components is described in McNicholas U.S. Pat. No. 3,278,447, which is incorporated in its entirety herein by reference. Specific examples of useful SCD products include that sold under the trademark Dura Klor⁷ by Rio Linda Chemical Company, Inc., and that sold under the trademark Anthium Dioxide⁷ by International Dioxide, Inc. An especially useful SCD is a product sold under the trademark Purite™ by Allergan, Inc. Other examples of oxidative preservative components includes peroxy components. For example, trace amounts of peroxy components stabilized with a hydrogen peroxide stabilizer, such as diethylene triamine penta(methylene phosphonic acid) or 1-hydroxyethylidene-1,1-diphosphonic acid, may be utilized as a preservative for use in components designed to be used in the ocular environment. Also, virtually any peroxy component may be used so long as it is hydrolyzed in water to produce hydrogen peroxide. Examples of such sources of hydrogen peroxide, which provide an effective resultant amount of hydrogen peroxide, include sodium perborate decahydrate, sodium peroxide and urea peroxide. It has been found that peracetic acid, an organic peroxy compound, may not be stabilized utilizing the present system. See, for example, Martin et al U.S. Pat. No. 5,725,887, the disclosure of which is incorporated in its entirety herein by reference.

Preservatives other than oxidative preservative components may be included in the formulations. The choice of preservatives may depend on the route of administration. Preservatives suitable for formulations to be administered by one route may possess detrimental properties which preclude their administration by another route. For nasal and ophthalmic formulations, preferred preservatives include quaternary ammonium compounds, in particular the mixture of alkyl benzyl dimethyl ammonium compounds and the like known generically as “benzalkonium chloride.” For formulations to be administered by inhalation, however, the preferred preservative is chlorbutol and the like. Other preservatives which may be used, especially for formulations to be administered rectally, include alkyl esters of p-hydroxybenzoic acid and mixtures thereof, such as the mixture of methyl, ethyl, propyl, butyl esters and the like which is sold under the trade name “Nipastat.”

Preferably, the present preservative components or components which assist in the preservation of the formulation, preferably the TCs therein, are effective in concentrations of less than about 1% (w/v) or about 0.8% (w/v) and may be 500 ppm (w/v) or less, for example, in the range of about 10 ppm(w/v) or less to about 200 ppm(w/v).

The formulation may further comprise a carrier. The carrier components useful in the present invention are selected to be non-toxic and have no substantial detrimental effect on the present formulations, on the use of the formulations or on the human or animal to whom the formulations are administered. In one embodiment, the carrier component is a liquid carrier component. A particularly useful liquid carrier component is that derived from saline, for example, a conventional saline solution or a conventional buffered saline solution. The liquid carrier preferably has a pH in the range of about 6 to about 9 or about 10, more preferably about 6 to about 8, and still more preferably about 7.5. The liquid medium preferably has an ophthalmically acceptable tonicity level, for example, of at least about 200 mOsmol/kg, more preferably in the range of about 200 to about 400 mOsmol/kg. In an especially useful embodiment, the osmolality or tonicity of the carrier component substantially corresponds to the tonicity of the fluids of the eye, in particular the human eye.

In one embodiment, the carrier components containing the EECs and the TCs may have viscosities of more than about 0.01 centipoise (cps) at 25° C., preferably more than about 1 cps at 25° C., even more preferably more than about 10 cps at 25°C. In a preferred embodiment, the formulation has a viscosity of about 50 cps at 25°C. and comprises a conventional buffer saline solution, a carboxymethylcellulose and a Brimonidine tartrate.

In order to insure that the pH of the liquid carrier component, and thus the pH of the formulation, is maintained within the desired range, the liquid carrier component may include at least one buffer component. Although any suitable buffer component may be employed, it is preferred to select such component so as not to produce a significant amount of chlorine dioxide or evolve significant amounts of gas, such as CO₂. It is preferred that the buffer component be inorganic. Alkali metal and alkaline earth metal buffer components are advantageously used in the present invention.

Any suitable ophthalmically acceptable tonicity component or components may be employed, provided that such component or components are compatible with the other ingredients of the liquid carrier component and do not have deleterious or toxic properties which could harm the human or animal to whom the present formulations are administered. Examples of useful tonicity components include sodium chloride, potassium chloride, mannitol, dextrose, glycerin, propylene glycol and mixtures thereof. In one embodiment, the tonicity component is selected from inorganic salts and mixtures thereof.

The present formulations may conveniently be presented as solutions or suspensions in aqueous liquids or non-aqueous liquids, or as oil-in-water or water-in-oil liquid emulsions. The present formulations may include one or more additional ingredients such as diluents, flavoring agents, surface active agents, thickeners, lubricants, and the like, for example, such additional ingredients which are conventionally employed in formulations of the same general type.

The present formulation in the form of aqueous suspensions may include excipients suitable for the manufacture of aqueous suspensions. Such excipients are suspending agents, for example, sodium carboxymethyl-cellulose, methylcellulose, hydroxypropylmethylcellulose, sodium alginate, polyvinylpyrrolidone, gun tragacanth and gun acacia; dispersing or wetting agents may be a naturally occurring phosphatide, for example, lecithin, or condensation products of ethylene oxide with long chain aliphatic alcohols, for example, heptadecaethylene-oxycetanol, or condensation products of ethylene oxide with partial esters derived from fatty acids and a hexitol such as polyoxyethylene sorbitol mono-oleate, or condensation products of ethylene oxide with partial esters derived from fatty acids and hexitol anhydrides, for example, polyoxyethylene sorbitan mono-oleate, and the like and mixtures thereof. Such aqueous suspensions may also contain one or more coloring agents, one or more flavoring agents and one or more sweetening agents, such as sucrose, saccharin, and the like and mixtures thereof.

The present formulations in the form of oily suspensions may be formulated in a vegetable oil, for example, olive oil, sesame oil or coconut oil, or in a mineral oil such as liquid paraffin. Such suspensions may contain a thickening agent, for example beeswax, hard paraffin or cetyl alcohol. Sweetening agents, such as those set forth above, and flavoring agents may be added to provide a palatable oral preparation.

The present formulations may also be in the form of oil-in-water emulsions. The oily phase may be a vegetable oil, for example, olive oil or arachis oil, or a mineral oil, for example, liquid paraffin, and the like and mixtures thereof. Suitable emulsifying agents may be naturally-occurring gums, for example, gum acacia or gun tragacanth, naturally-occurring phosphatides, for example, soya bean lecithin, and esters or partial esters derived from fatty acids and hexitol anhydrides, for example, sorbitan mono-oleate, and condensation products of the said partial esters with ethylene oxide, for example, polyoxyethylene sorbitan mono-oleate. The emulsions may also contain sweetening and flavoring agents.

The present formulations in the form of syrups and elixirs may be formulated with sweetening agents, for example, as described elsewhere herein. Such formulations may also contain a demulcent, and flavoring and coloring agents.

The specific dose level for any particular human or animal depends upon a variety of factors including the activity of the active component employed, the age, body weight, general health, sex, diet, time of administration, route of administration, rate of excretion, drug combination and the severity of the particular condition undergoing therapy.

The following non-limiting examples illustrate certain aspects of the present invention.

### EXAMPLE 1

A 45-year-old African American male receives a routine eye examination. The patient does not use any alcohol, or cigarettes, and has no systemic illnesses. He does not have any allergies to any medications. Ocular evaluation of his eyes reveals the following results. With his habitual spectacle correction, his best corrected visual acuities are 20/20 in both eyes. His pupils are found to be round, equally reactive to light, and without any presence of afferent pupillary defect. A slit lamp evaluation reveals the presence of grade 4 von Herrick angles in both eyes. His intraocular pressures are measured at 32 mmHg in both eyes (with the Goldmann tonometer at 3 pm). Dilated fundus evaluation reveals the presence of 0.7 round cup to disc ratio, without any presence of retinal fiber layer defect. A Humphrey C-20 automated threshold visual field test indicates the presence of generalized depression in both eyes. Gonioscopy evaluation indicates that the ciliary body is seen in all four quadrants. His blood pressure is 125/85 and his pulse rate is 60 beats/min. Because of the enlarged cup to disc ratio, elevated intraocular pressures, and the generalized depression of the threshold visual fields, he is diagnosed with open angle glaucoma. Timolol 0.5% 1 drop two times daily in both eyes is prescribed for two weeks. The patient is to return in two weeks for a follow-up evaluation. At the follow-up evaluation, he states that he has been compliant with the prescribed therapy. His intraocular pressure is measured at 28 mmHg at 1 pm with the Goldmann tonometer. His blood pressure is 118/86 and his pulse rate is 65 beats/min. Bromonidine-conjugate 0.2% 1 drop two times daily in both eyes is added to the treatment regimen. After another two weeks, the patient has a follow-up evaluation, during which, the patient's intraocular pressure measures 18 mmHg in the right eye, and 17 mmHg in the left eye. His blood pressure and pulse rate remain normal. The patient is re-evaluated every 3 months with the continuation of combination therapy. During subsequent examinations the intraocular pressure remains normal and no further changes in optic nerve head and threshold visual field are observed.

### EXAMPLE 2

A 34-year-old American Indian female comes to the emergency room complaining of dizziness, weakness, and fluctuations in vision. Her blood pressure is 100/60, and her heart rate is 120 beats/min. No murmurs or gallops are noted on auscultation, and a blood glucose dipstick test measures glucose levels exceeding 250 mg/dl. Insulin levels are absent. A diagnosis of type I diabetes is made and the patient is given a bolus of insulin. Ten minutes after the insulin injection, the patient feels “much better” but is still having visual disturbances. The patient is referred to the ophthalmology clinic. Examination reveals widespread dot and blot hemorrhages, macular edema, with microaneurysms and cotton wool spots throughout the retina. A diagnosis of moderate diabetic retinopathy with clinically significant macular edema secondary to diabetic complications is made. To prevent further ischemic neuronal damage due to diabetic vascular changes, 100 ng (insulin-like growth factor-1)-conjugate (IGF-1-conjugate) drops two times daily in both eyes is prescribed. For the next three days, this regimen is continued in addition to continuous control of her blood glucose with insulin drip. A reduction in macular edema and microaneurysms, and an improvement in vision are noted. The patient is discharged but is asked to continue the IGF-1-conjugate drops for an additional week while maintaining her insulin injections. At her 6-month follow-up examination, the patient states that she is in compliance with her treatment. The patient suffers no visual fluctuations, and diabetic retinopathy with clinically significant macular edema is resolved.

### EXAMPLE 3

A 17-year-old Asian male comes to a clinic complaining of severe pain, redness, tearing, and photophobia in his left eye. One day before, the patient was hit by a tree twig while playing football in the park. Severe conjunctival hyperemia with clear discharge is observed. A slit lamp exam of the left eye reveals a 0.5 mm gray/white corneal opacity with feathery border and positive sodium fluorescein (Na Fl) stain, hypopyon, and the presence of 2-3+ cells and flares in the anterior chamber. Intraocular pressure is also elevated (20 mmHg) by Goldman tonometry. A fungal corneal ulcer is diagnosed. The patient is administered 5% natamycin drops each hour during the day and every two hours at night and 5% homatropine four times each day for his left eye. The patient has a follow-up examination the next day and his symptoms have not improved. The patient is experiencing blurriness in the left eye. A slit lamp exam reveals extensions of the feathery borders on the cornea as well as an increased anterior chamber reaction. The extent of Na Fl stain is also enlarged. The patient is given 0.15% amphotericin B drops and 5% ketoconazole-conjugate drops once daily in both eyes. The next day, hyperemia is dramatically improved as well as visual acuity. 5% natamycin drops every two hours are administered around the clock, as well as 5% homatropine two times daily, and 5% ketoconazole-conjugate drops once daily. The patient is kept on the same regimen and has follow-up examinations daily for one week until signs and symptoms are completely resolved.

### EXAMPLE 4

A 20-year-old Caucasian female enters a clinic with complaints of pain, redness, and photophobia in her right eye. Gross observation reveals the presence of severe mucopurulent discharge. A slit lamp exam reveals a 1 mm corneal opacity with distinct margin and positive intense Na Fl staining. A positive 2+ anterior chamber reaction is present. The patient is asked about contact lens wear. She says that she has been wearing the same pair of disposable lenses, without proper cleaning, as extended wear lenses for the past three weeks due to studying for final exams. A bacterial corneal ulcer secondary to improper lens care and use is determined. She is instructed to discontinue all contact lens wear. The patient is given 1 drop fortified tobramycin-conjugate four times daily, 5% homatropine four times daily, and 2 drops 0.3% Ciloxan-conjugate every 3 hours for her right eye. The patient has a follow-up examination the next day. Improvement is noticed on visual inspection and slit lamp exam. The patient is experiencing a reduction in discharge and pain, and fewer cells are noted in the anterior chamber. The treatment regimen is tapered to 1 drop fortified tobramycin-conjugate two times daily and 5% homatropine two times daily. The next day, the patient's symptoms and signs are dramatically improved. The regimen is given for an additional 3 days resulting in a complete resolution of the corneal ulcer.

### EXAMPLE 5

A 30-year-old Caucasian male complains of gradual onset of floaters and progressive reduction of vision in both eyes, but without pain. The patient has been HIV+ for 7 years, and was recently hospitalized for Cryptococcal pneumonia. The patient is taking AZT, ddI, indinivir, and has been for the past three years, with moderate compliance. A blood workup reveals a CD+ count of 45 cells/mm³. An ophthalmic examination reveals the presence of cotton wool spots, with confluent areas of retinal opacities in the lower left quadrant of the left eye and upper right quadrant of the right eye. Granular hemorrhagic areas are noted throughout the retina, with a “brushfire” appearance. Visual field reveals scotomas in the upper right and lower left quadrants. These findings are all evidence of CMV retinitis, secondary to AIDS. Ganciclovir-conjugate 0.5% 1 drop four times daily in both eyes is prescribed. At a two-week follow-up examination, the patient states full compliance with the treatment protocol. The patient also states an improvement in vision with a reduced number of floaters. Ophthalmic examination reveals reduction in hemorrhages around the retinal vessels and cotton wool spots, with the retinal opacities remaining. At the one month follow-up, with the continuation of the same treatment regimen, complete remission of ocular manifestations of CMV retinitis becomes evident. The patient is advised to continue the ganciclovir-conjugate drops indefinitely.

Below is an example of a ganciclovir conjugate which may be used in accordance with this example. This conjugate preferably disassociates in the vitreous humor to release ganciclovir.

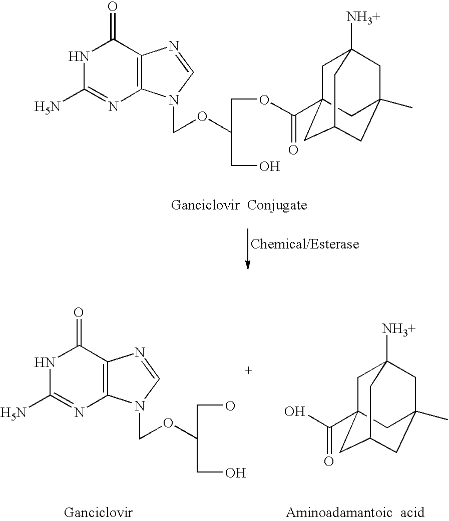

### EXAMPLE 6

### Bioavailability Study of Memantine

General disposition of topical ophthalmic 1-amino-3,5-dimethyladamantane hydrochloride (memantine) instillation was assessed by autoradiography. Briefly, albino and pigmented rabbits were dosed via topical ophthalmic instillation with a 0.74% aqueous isotonic memantine solution, pH 7.4. After dosing autoradiographic sections of the eye were acquired at 0.25, 0.5, 1, and 2 and 24 hours post dosing. The autoradiographic data clearly demonstrated that memantine was present in the posterior sclera, choroids and/or retina after topical dosing. Further, a qualitative assessment of residence time could be made from the data. The half-life of memantine in the posterior globe for both the albino and pigmented rabbits appeared relatively long. The intensity of the autoradiography in the pigmented tissues indicated that memantine bound to the ocular melanin.

Ocular tissue concentrations were also determined after oral and topical dosing. Rabbits were dosed topically with 35 μl of a 0.1% aqueous solution of memantine twice a day for 7 days. Another subset of rabbits was dosed orally with 2 mg/kg memantine for seven days. At the end of the dosing period ocular tissue concentrations were quantified. The retinal memantine concentrations from topical and oral dosing were essentially equivalent (107 ng/ml and 108 ng/ml, respectively). The tissue concentrations and autoradiographic data were sufficient evidence that topical dosing achieved comparable retinal concentrations to oral dosing. Moreover, the autoradiography indicated a trans-retinal route of penetration across the blood-retinal barrier from topical delivery. It was also shown in-vitro that melanin strongly bound to both natural and synthetic melanin.

Various references (including patents) have been cited. The disclosure of each of these references is incorporated in its entirety herein by reference.

While this invention has been described with respect to various specific examples and embodiments, it is to be understood that the invention is not limited thereto and that it can be variously practiced with the scope of the following claims.

What is claimed is:

1. A pharmaceutical conjugate comprising a therapeutic component and an efficacy enhancing component, the efficacy enhancing component has the general formula A: 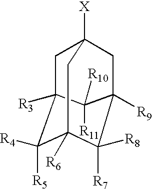
wherein X is 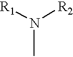
R1, R2, R3, R4, R5, R6, R7, R8, R9, R10 and R11 are independently an H, a C1-C10 hydrocarbon, or a linker.

2. A pharmaceutical conjugate of claim 1 wherein the therapeutic component and the efficacy enhancing component are directly joined by a covalent bond.

3. A pharmaceutical conjugate of claim 1 wherein the therapeutic component and the efficacy enhancing component are joined by a linker.

4. A pharmaceutical conjugate of claim 1 wherein R1 and R2 are Hs, and R3 is a linker.

5. A pharmaceutical conjugate of claim 1 wherein the efficacy enhancing component is a memantine.

6. A pharmaceutical conjugate of claim 1 wherein the linker is selected from the group consisting of: 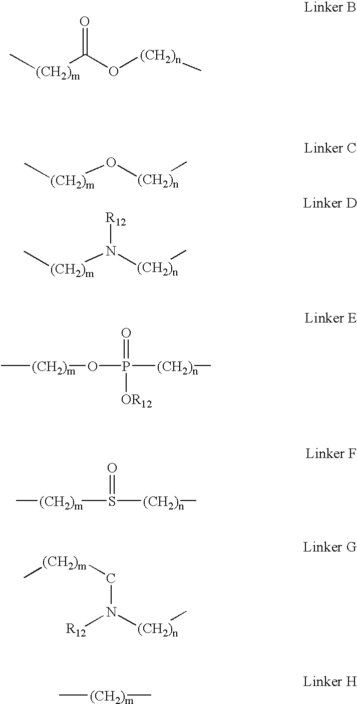
wherein R12 is an H or a C1-C10 hydrocarbon, m=0 to 10, and n=0 to 10.

7. A pharmaceutical conjugate of claim 1 wherein the therapeutic component is selected from the group consisting of NMDA antagonists, antibacterials, antihistamines, decongestants, antiinflammatories, antiparasitics, miotics, anticholinergics, adrenergics, antivirals, local anesthetics, antifungals, amoebicidals, trichomonocidals, analgesics, mydriatics, antiglaucoma drugs, carbonic anhydrase inhibitors, ophthalmic diagnostic agents, ophthalmic agents used as adjuvants in surgery, chelating agents, antineoplastics, antihypertensives, muscle relaxants, diagnostics, tyrosine kinase inhibitors and neuroprotectants.

8. A pharmaceutical conjugate of claim 1 wherein the therapeutic component is selected from the group consisting of quinoxaline, (2-imidozolin-2-ylamino) quinoxaline, 5-bromo-6-(2-imidozolin-2-ylamino) quinoxaline, derivatives thereof and mixtures thereof.

9. A pharmaceutical conjugate of claim 1 comprising a therapeutic component and a memantine, a linker joins the therapeutic component and the memantine.

10. A pharmaceutical conjugate of claim 1 comprising a timolol and a memantine, a linker joins the timolol and the memantine.

11. A pharmaceutical conjugate of claim 1 comprising a 5-bromo-6-(2-imidozolin-2-ylamino) quinoxaline and a memantine, a linker joins the 5-bromo-6-(2-imidozolin-2-ylamino) quinoxaline and the memantine.

12. A pharmaceutical conjugate of claim 1 wherein the therapeutic component and the efficacy enhancing component disassociate under physiological conditions.

13. A pharmaceutical conjugate of claim 1 being administered topically.

14. A pharmaceutical conjugate of claim 1 wherein the aqueous solubility, the partition coefficient and/or the affinity for melanin is higher than a compound comprising the same therapeutic component which is not joined to an efficacy enhancing component.

15. A pharmaceutical conjugate of claim 1 being a salt.

16. A pharmaceutical conjugate comprising a therapeutic component and an efficacy enhancing component, the efficacy enhancing component has the general formula A: 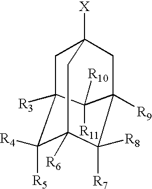
wherein X is 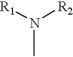
R1, R2, R3, R4, R5, R6, R7, R8, R9, R10 and R11 are independently an H, a C1-C10 hydrocarbon, or a linker; the linker is selected from the group consisting of: 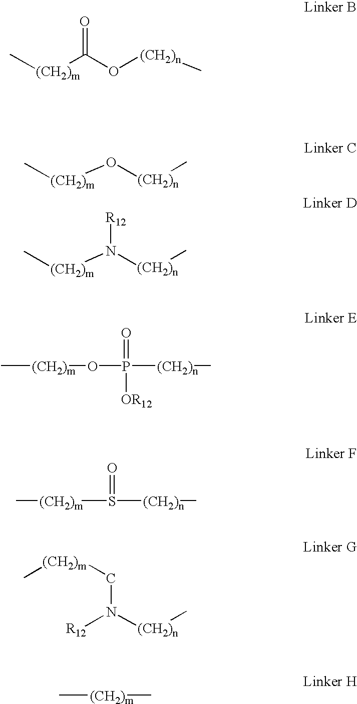
wherein R12 is an H or a C1-C10 hydrocarbon, m=0 to 25 10, and n=0 to 10.

17. A method for treating an ophthalmic condition, the method comprising the step of administering a pharmaceutical conjugate comprising a therapeutic component and an efficacy enhancing component, the efficacy enhancing component has the general formula I: 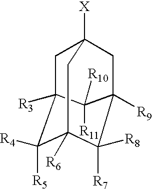
wherein X is an H or a 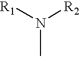
R1, R2, R3, R4, R5, R6, R7, R8, R9, R10 and R11 are independently an H, a C1-C10 hydrocarbon, or a linker.

18. A method of claim 17 wherein R1 and R2 are H, and R3 is a linker.

19. A method of claim 17 wherein the efficacy enhancing component is a memantine.

20. A method of claim 17 wherein the linker is selected from the group consisting of: 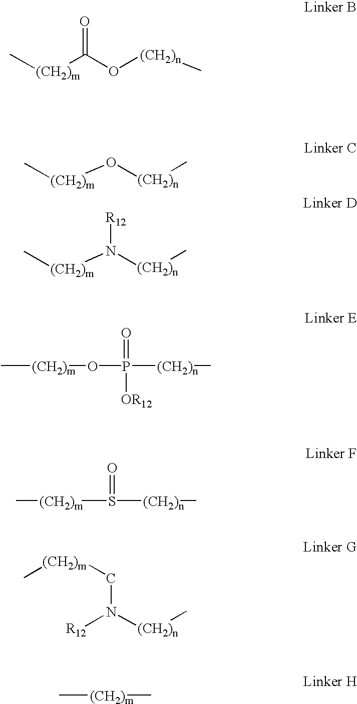
wherein R12 is an H or a C1-C10 hydrocarbon, m=0 to 10, and n=0 to 10.

21. A method of claim 17 wherein the therapeutic component is selected from the group consisting of NMDA antagonists, antibacterials, antihistamines, decongestants, antiinflammatories, antiparasitics, miotics, anticholinergics, adrenergics, antivirals, local anesthetics, antifungals, amoebicidals, trichomonocidals, analgesics, mydriatics, antiglaucoma drugs, carbonic anhydrase inhibitors, ophthalmic diagnostic agents, ophthalmic agents used as adjuvants in surgery, chelating agents, antineoplastics, antihypertensives, muscle relaxants, diagnostics, tyrosine kinase inhibitors and neuroprotectants.

22. The method of claim 17 wherein the route of administration is topical.

23. The method of claim 17 wherein the route of administration is oral, rectal, sublingual, nasal, and/or intravenous.
